(19) United States
(12) Patent Application Publication
(10) Pub. No.: US 2010/0130614 A1
(43) Pub. Date: May 27, 2010

(54) ADMINISTRATION OF ADAPALENE FOR MODULATING THE EXPRESSION OF IL-1ra

(75) Inventor: Brigitte DRENO, Nantes Cedex (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/468,641

(22) Filed: May 19, 2009

Related U.S. Application Data
(63) Continuation of application No. PCT/FR2007/052367, filed on Nov. 20, 2007.

(51) Int. Cl. A61K 31/192; A61P 17/00; A61P 17/10

(52) U.S. Cl. 514/569

(57) ABSTRACT

Adapalene or pharmaceutically acceptable salt thereof is administered to an individual in need of such treatment for modulating the expression of IL-1ra by the cells of acneic epidermis.

### CROSS-REFERENCE TO PRIORITY/PROVISIONAL APPLICATIONS

This application claims priority under 35 U.S.C. §119 of U.S. Provisional Application No. 60/859,967, filed Nov. 20, 2006, and is a continuation/national phase of PCT/FR 2007/052367, filed Nov. 20, 2007 and designating the United States (published in the French language on May 29, 2008 as WO 2008/062135 A1; the title and abstract were also published in English), each hereby expressly incorporated by reference in its entirety and each assigned to the assignee hereof.

### BACKGROUND OF THE INVENTION

1. Technical Field of the Invention

The invention present relates to the administration of adapalene for modulating the expression of IL-1ra (interleukin-1 receptor accessory protein), in particular in patients suffering from acne.

2. Description of Background and/or Related and/or Prior Art

Acne is a chronic disease associated with inflammation of the pilosebaceous follicle, under hormonal control, and which involves three well-defined stages (Cunliffe W J et al., Br. J. Dermatol., 2000: 142: 1084-1091; Gollnick H P et al., J. Dermatol., 1991: 18: 489-499).

The first stage, which generally begins at puberty, corresponds to the stimulation of production by the sebaceous glands, inducing hyperseborrhoea. The second stage corresponds to the formation of microcomedones considered to be the first elementary lesion of acne caused by anomalies in the proliferation, adhesion and differentiation of keratinocytes in the lower part of the sebaceous canal of the hair follicle.

The third stage corresponds to the formation of inflammatory acneic lesions in which Propionibacterium acnes (P. acnes), an anaerobic bacterium, plays an essential role.

The treatments employed against acne are intended to prevent the development of bacteria with antiseptics such as benzoyl peroxide or antibiotics, or to reduce sebum secretion (retinoids).

Employed only in the case of severe acne, the oral treatments can be broken down into three categories: antibiotics (tetracycline and erythromycin), retinoids (especially represented by isotretinoin) and hormone treatments combining an oestrogen and a progestogen.

### SUMMARY OF THE INVENTION

It has now surprisingly been demonstrated that adapalene has a comedolytic action and behaves as a stimulator of the expression of IL-1ra in the skin and makes it possible, in particular, to increase the expression thereof in the keratinocytes.

The present invention thus features administration of adapalene, or of a pharmaceutically acceptable salt thereof, for increasing the expression of IL-1ra and/or for inducing the increased expression of IL-1ra by the cells of the epidermis, and in particular by the keratinocytes.

This invention also features the formulation of adapalene, or of a pharmaceutically acceptable salt thereof, into medicaments useful for increasing the expression of IL-1ra and/or for inducing the increased expression of IL-1ra by the cells of acneic epidermis.

The administration of adapalene or of a pharmaceutically acceptable salt thereof is also useful for preventing the formation of comedones in patients in whom the cells of the acneic epidermis underexpress IL-1ra.

The medicament is preferably applied topically. Thus, it may be in the form of a gel, a lotion or a cream.

The present invention also features a regime or regimen for inducing an increase in the expression of IL-1ra by the cells of the epidermis of a patient requiring treatment, wherein adapalene or a pharmaceutically acceptable salt thereof is administered to said patient.

The present invention will now be more fully described in the detailed description which follows.

### BRIEF DESCRIPTION OF THE DRAWINGS

FIG. 1 indicates evaluation of the expression of IL-1ra in explants of healthy human skin,

FIGS. 2(a) and 2(b) indicate evaluation of the expression of IL-1ra in explants of healthy human skin originating from two groups of healthy donors,

FIG. 3 indicates evaluation of the expression of IL-1ra in explants of acneic skin, and

FIGS. 4(a) and 4(b) indicate evaluation of the amount of IL-1ra secreted in the culture supernatants of explants of healthy human skin (a) and of explants of acneic skin (b).

### DETAILED DESCRIPTION OF BEST MODE AND SPECIFIC/PREFERRED EMBODIMENTS OF THE INVENTION

The term “adapalene salts” means the salts formed with a pharmaceutically acceptable base, in particular inorganic bases such as sodium hydroxide, potassium hydroxide and aqueous ammonia, or organic bases such as lysine, arginine or N-methylglucamine.

The term “adapalene salts” means the salts formed with fatty amines such as dioctylamine and stearylamine.

Adapalene is a chemically stable derivative of naphthoic acid. The name of this derivative is the following: 6-[3-(1-adamantyl)-4-methoxyphenyl]-2-naphthoic acid.

Adapalene is a second-generation topical retinoid (like tazarotene) which binds selectively to the RARγ (found mainly in the epidermis) and RARβ (found mainly in the fibroblasts of the dermis) subtypes of the nuclear retinoic acid receptor (RAR), activating the genes responsible for cell differentiation. It does not, on the other hand, bind to the cellular retinoic acid binding proteins. However, clinically, adapalene also exhibits an activity on superficial inflammatory lesions (Millikan L E., Int. J. Dermatol., 2000: 39: 784-788). The mechanisms of the anti-inflammatory activity appear in particular to be related to the modulation of nonspecific immunity. Thus, adapalene inhibits the oxidative metabolism of arachidonic acid via the lipoxygenase pathway, the chemotaxis of polymorphonuclear neutrophils and the production of free radicals. It also inhibits the production of leukotrienes via the lipoxygenase pathway.

Adapalene is described in EP-0,199,636; a method for synthesizing this compound is described in EP-0,358,574.

The assignee hereof markets adapalene formulated at a concentration by weight of 0.1% in the form of an alcoholic lotion, an aqueous gel and a cream. These compositions are useful for the treatment of acne.

In the context of the present invention, a molecule has now been identified, expressed by the keratinocytes, which plays an essential role in the development of skin inflammation: IL-1ra.

IL-1 is a very active pro-inflammatory cytokine produced by monocytes and macrophages, but also by endothelial cells and lymphocytes. It will stimulate the production of prostaglandins, of NO and of chemokines. IL-1 is transcribed from three genes carried by chromosome 2 and which exhibit numerous homologies: IL-1alpha, IL-1beta and IL-1ra.

These 3 proteins bind to the IL-1 receptor, which comprises two types:
the receptor I is composed of two subunits, IL-1RI and IL-1RAcP the type II receptor, IL-1RII.

The IL-1 molecule binds to the two parts of the type I receptor and thus effects activation of its cytoplasmic TIR domain (analogous to the Toll-like receptor). IL-1ra binds only to the IL-1RI part and then prevents the activation of the intracellular signal. The effects of IL-1 are blocked. Binding to the type II receptor does not lead to any intracellular signal. The type I receptor is present on a very large number of cells, whereas the type II receptor is expressed mainly on neutrophils, monocytes and B lymphocytes.

II-1Ra is secreted by the liver as an acute-phase protein and therefore plays a role of inflammatory response modulator.

Nothing would therefore encourage those skilled in the art to suspect a mechanism of action for adapalene involving pro-inflammatory cytokine membrane receptors.

It has now been shown, in the context of the present invention, that adapalene induces an increase in the expression of IL-1ra by the cells of the epidermis, in particular the keratinocytes of inflammatory acneic lesions.

The term “increase” means a rise in, an overexpression of or an elevation of the level of expression of the IL-10 gene or an overproduction of the expression product thereof (mRNA, protein).

The increase in the expression of IL-1ra with adapalene is involved in the comedolytic action of adapalene. Specifically, since IL-1ra is an IL-1alpha receptor antagonist cytokine, it exerts anti-inflammatory effects at the level of the skin. It has now been demonstrated that, by blocking the IL-1alpha receptor, IL-1ra prevents the formation of comedones which would otherwise be induced by IL-1alpha.

This invention therefore features administration of a compound which modulates the expression of IL-1ra, such as adapalene or a pharmaceutically acceptable salt thereof, for increasing the expression of IL-1ra by the cells of the epidermis, and more particularly by the keratinocytes.

One particular embodiment of the invention features formulation of a compound which modulates the expression of IL-1ra, such as adapalene or a pharmaceutically acceptable salt thereof, into medicaments for increasing the expression of IL-1ra by the cells of acneic epidermis, in particular by the keratinocytes of a skin lesion, in particular of an inflammatory acneic lesion.

Another particular embodiment of the invention features formulation of a compound which modulates, more particularly stimulates, the expression of IL-1ra, such as adapalene or a pharmaceutically acceptable salt thereof, into medicaments for preventing the formation of comedones in patients in whom the cells of the acneic epidermis, in particular the keratinocytes, underexpress IL-1RA.

Preferably, the subject medicaments used herein are useful for treating acne, optimally, in patients in whom the cells of the acneic epidermis, in particular the keratinocytes, underexpress IL-1RA.

The present invention also features pharmaceutical compositions. The pharmaceutical compositions comprise a compound which modulates the expression of IL-1ra, such as adapalene or a pharmaceutically acceptable salt thereof, formulated into a physiologically acceptable medium.

Those skilled in the art can readily determine and prescribe the required amount of therapeutically active agent. For example, they may initiate therapy with doses of therapeutically active agent at levels below those required to obtain the desired therapeutic effect, and gradually increase the dosage until the desired effect is obtained.

In the case of adapalene, to provide an order of magnitude, the compositions according to the invention comprise from 0.001% to 5%, and advantageously from 0.01% to 1% by weight of adapalene, relative to the total weight of the composition, preferentially from 0.03% to 0.5%, preferably from 0.1% to 0.4% by weight of adapalene, even more preferentially 0.3% by weight of adapalene.

Such compositions may also comprise any additive normally used in the cosmetics or pharmaceutical field, such as propenetrating agents, wetting liquid surfactants, sequestering agents, antioxidants, sunscreens, preservatives, fillers, electrolytes, humectants, dyes, usual inorganic or organic bases or acids, fragrances, essential oils, cosmetic active agents, moisturizing agents, vitamins, essential fatty acids, sphingolipids, self-tanning compounds such as DHA, and agents for soothing and protecting the skin, such as allantoin. Of course, those skilled in the art will take care to select this or these optional additional compound(s), and/or the amount thereof, in such a manner that the advantageous properties of the compositions according to the invention are not, or are not substantially, impaired.

These additives may be present in the composition in a proportion of from 0 to 20% by weight relative to the total weight of the composition.

Such a composition may be administered by any known route, conventionally associated with the treatment (administering adapalene) of a dermatological disorder, i.e., topically, enterally, parenterally or ocularly.

Preferably, the treatment is administered topically.

The pharmaceutical composition is preferably in the form of a gel, a lotion or a cream.

Conventionally, the composition is in the form of an aqueous gel. The term “aqueous gel” means a composition containing, in an aqueous phase, a viscoelastic mass formed from colloidal suspensions (gelling agent).

Exemplary gelling agents include those of the polyacrylamide group, such as the sodium acryloyldimethyltaurate copolymer/isohexadecane/polysorbate 80 mixture marketed under the trademark Simulgel 600 by Seppic, the polyacrylamide/isoparaffin C13-14/laureth-7 mixture such as, for example, that marketed under the trademark Sepigel 305 by Seppic, the group of acrylic polymers coupled to hydrophobic chains, such as the PEG-15/decyl/SMDI copolymer marketed under the trademark Aculyn 44 (polycondensate comprising at least, as elements, a polyethylene glycol comprising 150 or 180 mol of ethylene oxide, decyl alcohol and methylenebis(4-cyclohexylisocyanate) (SMDI), at 35% by weight in a mixture of propylene glycol (39%) and water (26%)), the group of modified starches, such as the modified potato starch marketed under the trademark Structure Solanace, or else mixtures thereof.

The preferred gelling agents are from the polyacrylamide group, such as Simulgel 600 or Sepigel 305, or mixtures thereof.

The gelling agent as described above can be included at the preferred concentrations ranging from 0.1% to 15%, and more preferably ranging from 0.5% to 5%.

The medicaments and compositions as described above are useful for the treatment of inflammatory lesions of any type of acne, i.e., in particular, acne vulgaris, comedonal acne, polymorphous acne, acne rosacea, nodulocystic acne, acne conglobata, senile acne, secondary acne such as solar acne, acne medicamentosa, work-related acne or occupational acne.

### FIGURES OF DRAWINGS

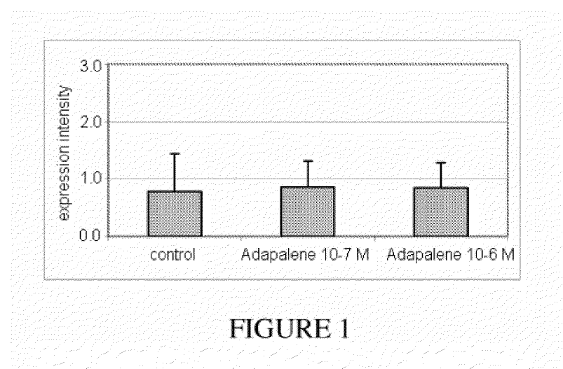

FIG. 1. Evaluation of the expression of IL-1ra in explants of healthy human skin, after incubating for 24 hours with 10⁻⁷ M and 10⁻⁶ M of adapalene or of a control medium (n=8 in each group).

Bars and error bars represent mean and SEM. *p<0.05 [adapalene versus control paired Wilcoxon test].

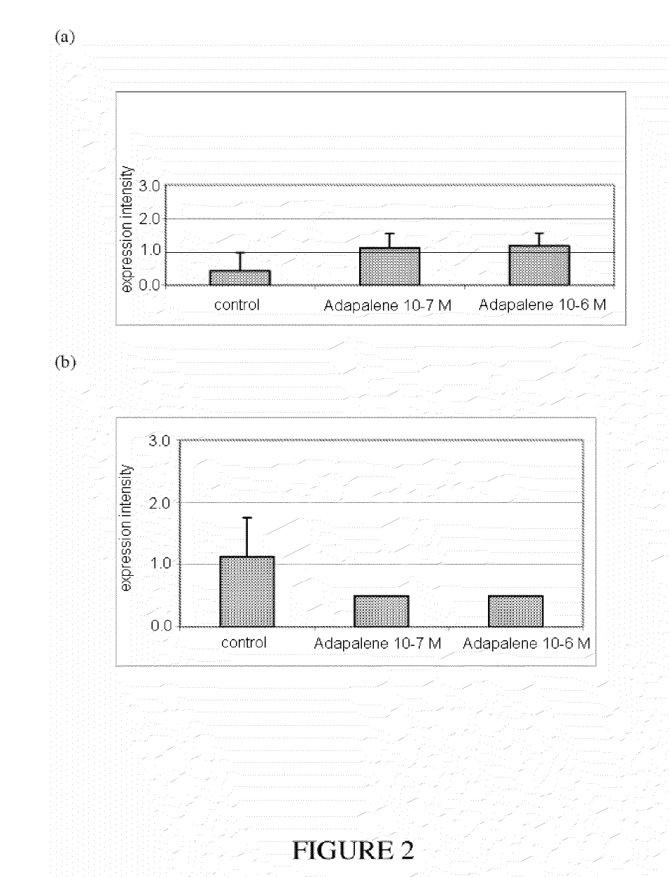

FIG. 2. Evaluation of the expression of IL-1ra in explants of healthy human skin originating from two groups of healthy donors, after incubation for 24 hours with 10⁻⁷ M and 10⁻⁶ M of adapalene or of control medium (n=4 in each group).

Bars and error bars represent mean and SEM. * p<0.05 [adapalene versus control paired Wilcoxon test].

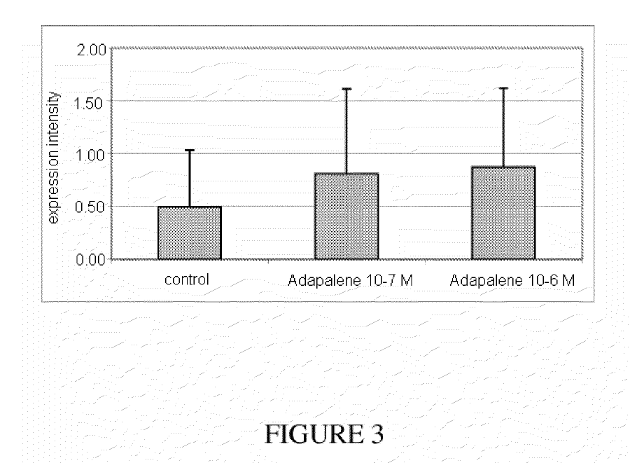

FIG. 3. Evaluation of the expression of IL-1ra in explants of acneic skin, after incubation for 24 hours with 10⁻⁷ M and 10⁻⁶ M of adapalene or of control medium (n=8 in each group).

Bars and error bars represent mean and SEM. *p<0.05 [adapalene versus control paired Wilcoxon test].

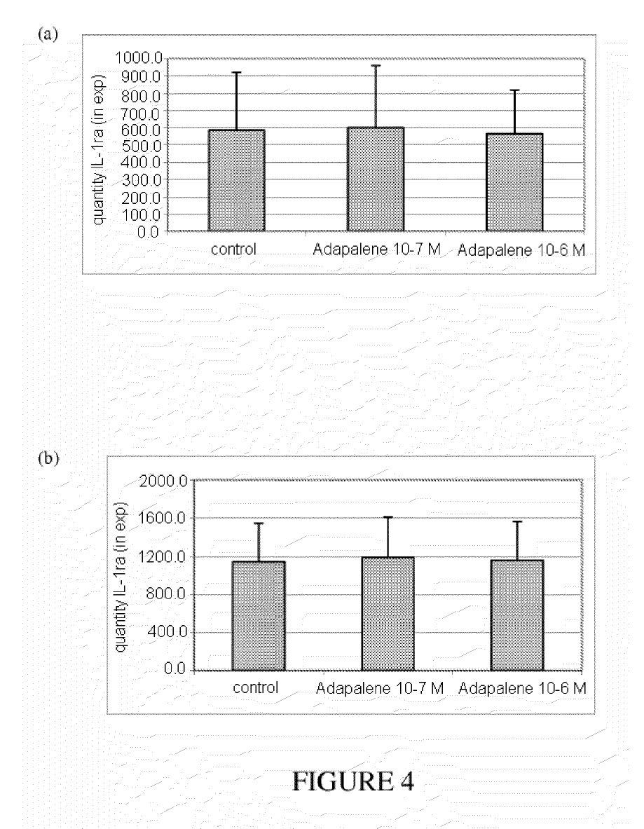

FIG. 4. Evaluation of the amount of IL-1ra secreted in the culture supernatants of explants of healthy human skin (a) and of explants of acneic skin (b), after incubation for 24 hours with 10⁻⁷ M and 10⁻⁶ M of adapalene or of control medium (n=8 in each group).

Bars and error bars represent mean and SEM. * p<0.05 [adapalene versus control paired Wilcoxon test].

Materials and Methods

Material:

The invention was carried out using biopsies taken from acneic individuals to be under conditions closest to the studies carried out in vivo.

Skin Biopsies:

Acneic Skin:

A skin biopsy (1×1.5 cm) was obtained from inflammatory papulae of lesions on the back of 8 acneic patients (having received no local treatment for 2 weeks or no general treatment for 1 month, or 3 months with isotretinoin). The fragments sampled were immediately placed in culture.

Healthy Skin:

Fragments of healthy skin from 8 healthy donors were obtained from mammary plasties (plastic surgery) or from a foreskin (infant surgery).

Explants of Healthy Skin or of Acneic Skin:

The fragments of healthy skin and of acneic skin were incubated at 37° C. in a humid atmosphere and in the presence of 5% CO₂ for 24 hours in KGM culture medium without hydrocortisone (PromoCell, Heidelberg, Germany). The culture medium was supplemented with 2 different concentrations of adapalene (Galderma, Sophia Antipolis): 10⁻⁷ M and 10⁻⁶ M. The culture medium alone served as control medium.

After incubation, the explants were removed from the culture medium and were then frozen in liquid nitrogen for the immunohistochemical study. The culture supernatants were frozen at −80° C. to evaluate the protein secretion using an ELISA assay technique.

Immunohistochemistry:

The expression of IL-1ra was evaluated by an immunoperoxidase technique in the explants of healthy skin from 8 healthy donors and of the acneic skin from 8 patients.

For this, cryostat sections 5 μm thick were cut from the skin explants, and were then fixed in acetone at 4° C. for 10 minutes. After saturation of the nonspecific sites with bovine albumin (BSA) for 30 minutes at ambient temperature, the slides were incubated for 30 minutes at ambient temperature and in a humid atmosphere in the presence of the corresponding primary antibody [anti-IL-1ra monoclonal antibody at 10 μg/ml (R&D Systems)] or of an irrelevant isotype antibody for the negative control (normal goat IgG at (R&D Systems), IgG2a monoclonal antibody (DAKO, Trappes, France), IgG1 monoclonal antibody (DAKO)).

After washing for 15 minutes in TBS/0.1% BSA/0.05% Tween 20, the slides were incubated with a biotinylated secondary antibody (DAKO) for 30 minutes at ambient temperature and in a humid atmosphere. After a further washing step, the slides were incubated for 30 minutes in the presence of streptavidin/peroxidise (DAKO) for 30 minutes at ambient temperature and in a humid atmosphere. After a final wash, the slides were incubated for 5 minutes in the presence of AEC (DAKO), a substrate for peroxidise, and then counterstained with Mayer's hemalun. The slides were read under a microscope.

The slides were read under a Leitz microscope by 2 investigators.

A semi-quantitative scale was used to define the intensity of the labeling: 0=none, 1=weak, 2=medium, 3=strong.

A mean was established on 8 healthy donors or 8 acneic patients in each of the “control”, “10⁻⁷ M adapalene” and “10⁻⁶ M adapalene” groups.

ELISA Assay:

The IL-1ra protein secretion (patients only) in the culture supernatants of the explants of acneic skin and of healthy skin, incubated in the presence or absence of adapalene, was evaluated by means of an ELISA assay using the IL-1ra Cytoscreen detection kit (Biosource Europe S.A., Nivelles, Belgium) and according to the supplier's instructions.

A mean was established on 8 healthy donors or 8 acneic patients in each of the “control”, “10⁻⁷ M adapalene” and “10⁻⁶ M adapalene” groups.

Statistical Analysis:

The paired Wilcoxon test was employed for the statistical analysis, and p<0.05 (by comparison with the control medium) was considered to be significant.

To further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Measurement of the Expression of IL-1ra on Healthy Skin and on Acneic Skin by Immunohistochemistry

Immunohistochemistry: Healthy Skin (FIG. 1):

IL-1ra is very weakly-to-weakly expressed in the epidermis of the healthy donors, its expression intensity being on average 0.8 (+/−0.7).

The mean calculated on 8 healthy donors does not demonstrate a modulatory effect of adapalene on the expression of IL-1ra in the epidermis of the healthy donors. The intensity of expression of this cytokine is in fact 0.9 (+/−0.5) with 10⁻⁷ M with adapalene and 0.8 (+/−0.4) with 10⁻⁶ M of adapalene.

The Modulatory Effect of Adapalene is Exerted in 2 Ways [cf. FIGS. 2(a) and (b)]: an inducing effect when the expression in the control medium is weak [FIG. 2(a)].

In the 1st subgroup of healthy donors, the intensity of expression of IL-1ra in fact goes from 0.4 (+/−0.6) in the control medium to 1.1 (+/−0.4) with 10⁻⁷ M of adapalene, and then to 1.2 (+/−0.4) with 10⁻⁶ M of adapalene;
an inhibitory effect when the expression in the control medium is greater [FIG. 2(b)].

In the 2nd subgroup of healthy donors, the intensity of expression of IL-1ra in fact goes from 1.1 (+/−0.6) in the control medium to 0.5 (+/−0.0) with 10⁻⁷ M and 10⁻⁶ M of adapalene.

Immunohistochemistry: Acneic Skin (FIG. 3):

IL-1ra is very weakly expressed in the epidermis of the acneic patients: the intensity of expression is on average 0.5 (+/−0.5).

The intensity of expression of IL-1ra is therefore similar in the skin of the healthy donors and in the acneic skin.

Adapalene increases the expression of this cytokine in the epidermis of the acneic patients. This effect is dose-dependent and at its maximum at the concentration of 10⁻⁶ M. The intensity of expression is in fact 0.8 (+/−0.8) with 10⁻⁷ M of adapalene and 0.9 (+/−0.7) with 10⁻⁶ M of adapalene.

### Example 2

### Measurement of the Expression of IL-1ra on Healthy Skin and on Acneic Skin by ELISA Assay

ELISA Assay: Culture supernatants of explants of healthy skin [FIG. 4(a)]:

The amount of IL-1ra secreted in the culture supernatants of the explants of healthy skin, incubated with the control medium, is on average 585 pg (+/−337) (mean established on 8 healthy donors).

In the presence of 10⁻⁷ M adapalene, the amount of IL-1ra secreted in the culture supernatants is on average 600 pg (+/−362), i.e., an increase of 2.6% compared with the control medium, and 564 pg (+/−255) in the presence of 10⁻⁶ M adapalene, i.e., a decrease of 3.5% compared with the control medium.

ELISA Assay: Culture Supernatants of Explants of Acneic Skin [FIG. 4(b)]:

The amount of IL-1ra secreted in the culture supernatants of the explants of acneic skin, incubated with the control medium, is on average 1145 pg (+/−404).

The secretion of IL-1ra is therefore, on average, higher in the acneic patients by comparison with the healthy donors.

In the presence of 10⁻⁷ M adapalene, the secreted amount of IL-1ra is on average 1196 pg (+/−422), i.e., an increase of 4.4% compared with the control medium, and 1161 pg (+/−409) in the presence of 10⁻⁶ M adapalene, i.e., an increase of 1.4% compared with the control medium.

The results of the examples show that adapalene (at the 2 concentrations used) increases the expression of IL-1ra in the epidermis of acneic patients. No significant modulation is observed in the healthy donors, but two subpopulations emerge.

The increase in the expression of IL-1ra with adapalene is involved in the comedolytic action of adapalene. Specifically, IL-1ra is an IL-1 alpha receptor antagonist cytokine which exerts anti-inflammatory effects on the skin (Suh D et al., 2002). By blocking the IL-1alpha receptor, IL-1ra prevents the formation of comedones which would otherwise be induced by IL-1alpha.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for modulating the expression of IL-1ra by the cells of the epidermis of an individual in need of such treatment, comprising administering to such individual a thus effective amount of adapalene or pharmaceutically acceptable salt thereof.

2. The regime or regimen as defined by claim 1, said cells of the epidermis comprising keratinocytes.

3. A regime or regimen for inducing the increased expression of IL-1ra by the cells of acneic epidermis of an individual in need of such treatment, comprising administering to such individual a thus effective amount of adapalene or pharmaceutically acceptable salt thereof.

4. A regime or regimen as defined by claim 3 for the treatment of acne vulgaris, comedonal acne, polymorphous acne, acne rosacea, nodulocystic acne, acne conglobata, senile acne, secondary acne, solar acne, acne medicamentosa, work-related acne and/or occupational acne, comprising administering to such individual in need of such treatment, a thus effective amount of adapalene or pharmaceutically acceptable salt thereof.

5. A regime or regimen for preventing the formation of comedones in an individual wherein the cells of the acneic epidermis underexpress IL-1ra, comprising administering to such individual a thus effective amount of adapalene or pharmaceutically acceptable salt thereof.

6. The regime or regimen as defined by claim 1, comprising topically applying such effective amount of adapalene or pharmaceutically acceptable salt thereof, formulated into a physiologically acceptable medium therefor, onto the skin of such individual.

7. The regime or regimen as defined by claim 3, comprising topically applying such effective amount of adapalene or pharmaceutically acceptable salt thereof, formulated into a physiologically acceptable medium therefor, onto the skin of such individual.

8. The regime or regimen as defined by claim 5, comprising topically applying such effective amount of adapalene or pharmaceutically acceptable salt thereof, formulated into a physiologically acceptable medium therefor, onto the skin of such individual

9. The regime or regimen as defined by claim 6, said adapalene or salt formulation thereof comprising a gel, a lotion or a cream.

10. A regime or regimen for inducing an increase in the expression of IL-1ra by the cells of the epidermis of an individual in need of such treatment, comprising administering to such individual a thus effective amount of adapalene or pharmaceutically acceptable salt thereof.
